(19) United States
(12) Patent Application Publication
(10) Pub. No.: US 2016/0208330 A1
(43) Pub. Date: Jul. 21, 2016

(54) IDENTIFYING MARKERS OF CALORIC RESTRICTION AND CALORIC RESTRICTION MIMETICS

(71) Applicant: NSE Products, Inc., Provo, UT (US)

(72) Inventors: Angela Mastaloudis, Holladay, UT (US); Steve Wood, Santaquin, UT (US); Tomas Alberto Prolla, Madison, WI (US); Jamie Louis Barger, Verona, WI (US); Richard Weindruch, Madison, WI (US); Joseph Chang, Sandy, UT (US)

(73) Assignee: NSE Products, Inc., Provo, UT (US)

(21) Appl. No.: 15/001,025

(22) Filed: Jan. 19, 2016

Related U.S. Application Data
(63) Continuation of application No. 13/525,230, filed on Jun. 15, 2012, now abandoned.
(60) Provisional application No. 61/497,476, filed on Jun. 15, 2011.

(51) Int. Cl. C12Q 1/68

(52) U.S. Cl. CPC C12Q 1/6883; C12Q 2600/158; C12Q 2600/16

(57) ABSTRACT

Markers of caloric restriction (CR) can be identified in a selected tissue by exposing an animal to CR conditions and selecting one or more genes differentially expressed in response to CR conditions in multiple subject groups. A candidate compound can be screened for likely ability to mimic the effects of CR when administered to an animal by comparing the tissue levels of expression products of the genes in animals treated with the candidate compound to those of animals subjected to CR.

### RELATED APPLICATIONS

This is a continuation of U.S. patent application Ser. No. 13/525,230, filed Jun. 15, 2012, which claims the benefit of U.S. Provisional Patent Application No. 61/497,476, filed on Jun. 15, 2011, each of which are incorporated in their entirety herein by reference.

### GOVERNMENTAL RIGHTS

This invention was made with government support under Grant No. 1 R43AG034833-01A1 awarded by the National Institute on Aging of the National Institutes of Health. The government has certain rights in the invention.

### FIELD OF THE INVENTION

The present invention relates generally to methods for identifying universal biomarkers of caloric restriction, including tissue-specific universal biomarkers of caloric restriction. In particular, the present invention provides robust panels of genes which undergo changes in expression with caloric restriction, and use of these universal biomarkers to identify nutrients, drugs, or other functional ingredients that can elicit the beneficial effects of caloric restriction (i.e., “caloric restriction mimetics”).

### BACKGROUND

When started either early in life or at middle age, restriction of caloric intake (CR) below ad libitum levels has been shown to increase lifespan in multiple species, including mammals such as rodents, and to prevent or delay the onset of many age-related conditions. Indeed, clinical trials have been initiated to test the ability of CR to improve health parameters in humans. However, the social, biological, and psychological consequences of food deprivation are not compatible with widespread implementation of this dietary regimen. For this reason, research has focused on the identification of substances that are capable of mimicking the beneficial effects of CR in the absence of reductions in caloric intake. Efforts have been directed toward identifying compounds that mimic one or more physiological or biochemical effects of CR, including finding compounds that can mimic the global gene expression profile of CR after exposure of animals or cells to these agents. In connection with the latter, methods to identify compounds that mimic CR based on a global alteration in gene expression profiles have been disclosed (Spindler et al., U.S. Pat. No. 6,406,853).

Despite the availability of such approaches, no universal, tissue-specific panels of CR biomarkers in the mouse models tested have been identified. Because different mouse strains have unique genetic, metabolic and physiological characteristics, it is unlikely that any given gene expression change in response to CR in any particular mouse strain will be reproduced in other mouse strains or organisms. Thus, there is a lack of useful markers identified to date.

### SUMMARY OF THE INVENTION

The technology set forth in the present disclosure overcomes shortfalls of prior efforts to identify Caloric Restriction (CR) biomarkers by identifying universal CR biomarkers—that is, markers that consistently correlate to a CR response across multiple different, genetically diverse, strains of animals. Identification of a panel of universal CR biomarkers allows the rapid screening of compounds that mimic CR, independent of animal strain or breed, and also without the need of global gene expression profiling.

In some embodiments, the present disclosure provides systems and methods for identifying robust and universally applicable gene expression markers of CR in specific tissues. One embodiment provides a gene panel of polynucleotides that are differentially expressed in a tissue in response CR. Particular embodiments include genes from murine, canine, feline, or human tissues. In another aspect, the gene panel includes genes from any of liver tissue, heart tissue, lung tissue, brain tissue, epithelial tissue, connective tissue, white adipose, skeletal muscle, blood, nervous tissue, urine, and saliva.

In some embodiments, genes assessed are one or more genes found in Table 1, Table 2, Table 3, Table 4, Table 5, Table 6 or any combination thereof. In some embodiments, the genes in the panel exhibit changes in gene expression between CR subjects and control subjects. In some embodiments the genes in the panel are selected because of their validation across a variety of animal models of CR.

In some embodiments, the technology provides a probe for detecting differential expression of universal markers of CR in a tissue that can include a polynucleotide that hybridizes a gene that is a universal marker of CR, or a polypeptide binding agent that binds to a polypeptide encoded by such a gene. In another embodiment, a composition includes two or more (e.g., 3 or more, 5 or more, 10 or more, 20 or more, 50 or more, etc.) polynucleotide or polypeptide probes. In more particular aspects, the polynucleotides are from heart tissue or skeletal muscle or white adipose tissue.

In one embodiment, a kit can include an amplification oligonucleotide that specifically hybridizes a gene listed in Tables 1 through 6 or a fragment thereof; and a labeled probe comprising a polynucleotide that specifically hybridizes a gene encoding proteins listed in Tables 1 through 6 or a fragment thereof. In a particular embodiment, the probe is bound to a substrate (e.g. as part of an array).

In some embodiments, the invention provides a method for measuring the effect of a candidate compound to mimic CR by determination of the expression profile of one or more genes differentially expressed in selected tissues of multiple animal strains.

The present disclosure arises from the inventors' development of a method for identifying a robust set of universal biomarkers of CR in selected tissues. In an embodiment, a method of identifying tissue-specific universal markers of caloric restriction (CR) in a selected tissue includes steps of exposing subjects belonging to a plurality of subject groups to CR conditions, and selecting one or more genes differentially expressed in response to CR in subjects from a multiplicity of the subject groups. The genes selected can be differentially expressed in at least two, or three, or more of the subject groups. In a particular embodiment the selected gene is differentially expressed in 50% or more of the subject groups tested. In accordance with the embodiment, subject groups can include murine groups, canine, groups, feline groups, or human groups.

In some embodiments, the present invention provides a method of assessing whether a given condition or candidate compound is likely to be effective in mimicking CR or one or more benefits of CR mimicry in a subject. The method can include exposing a first subject to CR, measuring the level of expression products of two or more genes in a sample of tissue from the first subject to obtain a CR expression profile, administering the candidate compound to a second subject, measuring the expression products in a sample of the tissue from the second subject, and comparing the levels to determine a degree to which the candidate compound mimics CR. Any of microarray analysis, reverse transcriptase PCR, quantitative reverse transcriptase PCR, or hybridization analysis can be used to measure expression products (e.g. mRNA) in the samples. A plurality of candidate compounds so assessed can be ranked based on the degree to which each mimics CR.

### BRIEF DESCRIPTION OF THE DRAWINGS

FIG. 1 is a set of graphs showing the body weights of mice in control groups and groups subjected to CR.

### DETAILED DESCRIPTION OF EMBODIMENTS

### Definitions

The terms “administration,” and “administering” refer to the manner in which a substance is presented to a subject. Administration can be accomplished by various art-known routes such as oral, parenteral, transdermal, inhalation, implantation, etc. Thus, an oral administration can be achieved by swallowing, chewing, sucking of an oral dosage form comprising the drug, or by ingesting a liquid or semi-liquid form e.g. via drinking or gavage. Parenteral administration can be achieved by injecting a drug composition intravenously, intra-arterially, intramuscularly, intrathecally, or subcutaneously, etc. Transdermal administration can be accomplished by applying, pasting, rolling, attaching, pouring, pressing, rubbing, etc., of a transdermal preparation onto a skin surface. These and additional methods of administration are well-known in the art.

The term “condition,” as used herein, is defined as any external factors that can be applied or administered to a subject. This term refers to compounds which may be administered to the subject, environmental factors which can be applied to the subject, stimuli which might affect the subject, etc. The condition may be qualitative or quantitative. Thus, this term includes pharmaceuticals, food supplements, diet regimen, health regimen, dietary supplements, nutraceuticals, environment, food, emotional stimuli, psychological stimuli, physical stimuli, genetic modification, etc.

As used herein, the term “tissue” means an aggregate of cells, together with intercellular substances, that forms a material. The cells may all be of a particular type, or may be of multiple cell types. The tissue may be any of the types of animal tissue, selected from, but not limited to: epithelial tissue, connective tissue, muscle tissue, blood, or nervous tissue. The tissue may come from any animal (e.g., human, mouse, etc.).

The term “oligonucleotide,” as used herein, is defined as a molecule comprised of two or more ribonucleotides, preferably more than three. Its exact size will depend upon many factors which, in turn, depend upon the ultimate function and use of the oligonucleotide.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1 methylpseudouracil, 1-methylguanine, 1-methylinosine, 2,2-dimethylguanine, 2-methyladenine, 2-methylguanine, 3-methyl cytosine, 5-methyl cytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil 5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “gene panel” and variants thereof refer to a group of identified genes, and particularly a group that are selected based on some common property or characteristic. For example a gene panel can comprise a plurality of genes found to be modified by some treatment or environmental factor (e.g. a caloric restriction regimen). In accordance with this usage, the term “panel” can be referred to by other names that indicate a grouping of genes such as a “cluster”, a “signature”, a “supermarker”, a “pattern”, or the like.

The term “expression” as used herein can be used to refer to transcription, translation, or both. Accordingly, “expression products” refers to products of transcription (e.g. mRNA), as well as products of translation (e.g. polypeptides).

As used herein, the term “changes in levels of gene expression” refers to higher or lower levels of gene expression (e.g., mRNA or protein expression) in a test subject (e.g., CR subject or subject exposed to test conditions) relative to the level in a control subject.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

The term “standard hybridization conditions” refers to salt and temperature conditions substantially equivalent to 5 times saline-sodium-citrate (SSC) buffer and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “amplification oligonucleotide” refers to an oligonucleotide that hybridizes to a target nucleic acid, or its complement, and participates in a nucleic acid amplification reaction. An example of an amplification oligonucleotide is a “primer” that hybridizes to a template nucleic acid and contains a 3′ OH end that is extended by a polymerase in an amplification process. Another example of an amplification oligonucleotide is an oligonucleotide that is not extended by a polymerase (e.g., because it has a 3′ blocked end) but participates in or facilitates amplification. Amplification oligonucleotides may optionally include modified nucleotides or analogs, or additional nucleotides that participate in an amplification reaction but are not complementary to or contained in the target nucleic acid. Amplification oligonucleotides may contain a sequence that is not complementary to the target or template sequence. For example, the 5′ region of a primer may include a promoter sequence that is non-complementary to the target nucleic acid (referred to as a “promoter-primer”). Those skilled in the art will understand that an amplification oligonucleotide that functions as a primer may be modified to include a 5′ promoter sequence, and thus function as a promoter-primer. Similarly, a promoter-primer may be modified by removal of, or synthesis without, a promoter sequence and still function as a primer. A 3′ blocked amplification oligonucleotide may provide a promoter sequence and serve as a template for polymerization (referred to as a “promoter-provider”).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i. e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to at least a portion of another oligonucleotide of interest. A probe may be single-stranded or double-stranded. Probes are useful in the detection, identification and isolation of particular gene sequences. It is contemplated that any probe used in the present invention will be labeled with any “reporter molecule,” so that it is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is present in a form or setting that is different from that in which it is found in nature. In contrast, non-isolated nucleic acids include nucleic acids such as DNA and RNA found in the state in which they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the terms “subject” and “patient” refer to any animal, such as a mammal like a dog, cat, bird, livestock, mouse, rat, and a human.

The phrases “candidate compound” or “candidate substance” refer to any chemical entity, pharmaceutical, drug, and the like that can be used to treat or prevent a disease, illness, sickness, or disorder of bodily function, or otherwise alter the physiological or cellular status of a sample, such as opposing aging. Test compounds comprise both known and potential therapeutic compounds. A test compound can be determined to be therapeutic by screening using the screening methods of the present invention.

As used herein, the term “food material” refers to any food type fed to humans or non-human animals. Food material includes food components (such as dough, flakes), food intermediates (a transitional step used in making a product or component) and food ingredients. Food material may be material of plant, fungal, or animal origin or of synthetic sources. Food material may contain a body nutrient such as a carbohydrate, protein, fat, vitamin, mineral, fiber, cellulose, etc.

As used herein, the term “nutraceutical” refers to any compounds or chemicals that can provide dietary or health benefits when consumed by humans or animals. Examples of nutraceuticals include vitamins, minerals, phytonutrients and others. The intent of nutraceuticals is to impart health benefits or desirable physiological effects that may not be associated with food.

The term “pharmaceutical agent or drug” as used herein refers to a chemical compound or composition capable of inducing a therapeutic effect when properly administered to a patient. Other chemistry terms herein are used according to conventional usage in the art, as exemplified by The McGraw-Hill Dictionary of Chemical Terms (Parker, S., Ed., McGraw-Hill, San Francisco (1985), incorporated herein by reference).

As used herein, the term “dietary supplement” refers to a product that is intended to supplement the diet that bears or contains one or more dietary ingredients including, but not limited to: a vitamin, a mineral, a micronutrient, a phytonutrient, an herb or other botanical, an amino acid, a dietary substance for use by man to supplement the diet by increasing the total daily intake, or a concentrate, metabolite, constituent, extract, or combinations of these things. Similar definitions exist in other parts of the world, e.g. in Europe. Different denominations concerning “dietary supplements” or similar food products are used around the world, such as “food supplements”, “nutraceuticals”, “functional foods” or simply “foods”. In the present context the term “food supplement” covers any such denomination or definition.

The term “genetic modification” as used herein refers to the stable or transient alteration of the genotype of a precursor cell by intentional introduction of exogenous DNA. DNA may be synthetic, or naturally derived, and may contain genes, portions of genes, or other useful DNA sequences. The term “genetic modification” as used herein is may also include naturally occurring alterations such as that which occurs through natural viral activity, natural genetic recombination, or the like.

As used herein, the term “environmental conditions” is defined to include one or more physical aspects of the environment. Environmental conditions may include any external factor which may or may not affect a subject (temperature, barometric pressure, gas concentrations, oxygen levels, radiation, air born particulates, etc.).

The term “diet regimen” refers to the food materials, ingredients, or mixture of ingredients including water, which is consumed by an animal subject over time. The term “diet regimen” may take into account the specific food materials consumed, variety of food materials, volume consumed, sources of food materials, frequency of feeding, time of feeding, etc.

The term “health regimen” refers to the daily activities of an animal subject, which may affect the subjects overall health, over time. The term “health regimen” may take into account the diet regimen, the use of supplements, the use of pharmaceuticals, exercise, sleep/rest, stress, etc.

The terms “formulation” and “composition” may be used interchangeably herein.

Concentrations, amounts, solubilities, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a concentration range of 0.1 to 5 ng/ml should be interpreted to include not only the explicitly recited concentration limits of 0.1 ng/ml and 5 ng/ml, but also to include individual concentrations such as 0.2 ng/ml, 0.7 ng/ml, 1.0 ng/ml, 2.2 ng/ml, 3.6 ng/ml, 4.2 ng/ml, and sub-ranges such as 0.3-2.5 ng/ml, 1.8-3.2 ng/ml, 2.6-4.9 ng/ml, etc. This interpretation should apply regardless of the breadth of the range or the characteristic being described.

As used herein, the term “about” means that dimensions, formulations, parameters, and other quantities and characteristics are not and need not be exact, but may be approximated and/or larger or smaller, as desired, reflecting tolerances, conversion factors, rounding off, measurement error and the like and other factors known to those of skill. Further, unless otherwise stated, the term “about” shall expressly include “exactly,” consistent with the discussion above regarding ranges and numerical data.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The present invention relates generally to methods for identifying conditions which mimic the metabolic effects of CR on an organ-specific basis. In particular, the present invention provides a panel of genes which undergo changes in expression with CR. This panel of genes provides markers for CR. The panel can be used to probe for conditions (e.g. pharmaceuticals, therapies, foods, supplements, environmental factors, etc.) which have the effect of mimicking CR.

Certain exemplary embodiments for practicing the invention are described in more detail below. The invention is not limited to these particular embodiments.

Assessment of Gene Expression

A wide variety of techniques may be used to assess gene expression of the markers of the present invention. Exemplary methods, kits, and reagents are described herein.

In some embodiments microarrays are used to assess marker expression. It is contemplated that the microarrays have a number of different oligonucleotides that have specificity for genes associated with CR and identified in Tables 1-3, attached to the surface of the solid support. It is contemplated that, in some embodiments, samples are prepared from tissue RNA samples of test subjects (e.g., subjects under a condition to be compared with CR for its effect upon aging) and, optionally, control subjects, and the prepared samples are applied to the microarrays for hybridization. It is contemplated that the differential hybridization of the test samples relative to the control samples or the amount of expression of the test sample as compared to a pre-established control value (e.g. from an expression profile obtained under CR) identifies the effect of the tested condition on aging.

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as a gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink-jet printing; or, electrochemistry on microelectrode arrays.

Southern and Northern blotting may also be used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

Genomic DNA and mRNA may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reverse transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPaS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

Non-amplified or amplified nucleic acids can be detected by any conventional means. For example, in some embodiments, nucleic acids, from a panel selected from Tables 1-3, are detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

Data Analysis

In some embodiments, a computer-based analysis program is used to translate the raw data generated by the detection assay (e.g., the presence, absence, or amount of expression of a panel of genes selected from a group consisting of the genes listed in Tables 1-6) into data of predictive value for a clinician or researcher. The user can access the predictive data using any suitable means. Thus, in some preferred embodiments, the present invention provides the further benefit that the user, who may not be trained in genetics or molecular biology, need not understand the raw data. The data are presented directly to the user in its most useful form. The user is then able to immediately utilize the information in order to optimize the care of the subject (or for themselves if the user is the subject).

The present invention contemplates any method capable of receiving, processing, and transmitting the information to and from laboratories conducting the assays, information providers, medical personal, and subjects. For example, in some embodiments of the present invention, a sample (e.g., a biopsy or a blood or serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication system). Once received by the profiling service, the sample is processed and a profile is produced (i.e., expression data), specific for the diagnostic or prognostic information desired for the subject.

The profile data are then prepared in a format suitable for interpretation by a user. For example, rather than providing raw expression data, the prepared format may represent a likelihood (e.g., likelihood that the tested condition mimics CR) for the subject, along with recommendations for particular treatment options. The data may be displayed to the user by any suitable method. For example, in some embodiments, the profiling service generates a report that can be printed for the user (e.g., at the point of care) or displayed to the user on a computer monitor.

In some embodiments, the information is first analyzed at the point of care or at a regional facility. The raw data are then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may choose further intervention or counseling based on the results. In some embodiments, the data are used for research use. For example, the data may be used to further optimize the inclusion or elimination of markers as useful indicators of a particular condition or stage of disease.

In some embodiments, the methods described herein can be used to create a panel of genes for which CR results in a change in expression. The genes in the panel can be selected according to further criteria, including but not limited to magnitude of change, direction or sign of change, level of statistical significance, robustness of the change across subject groups. “Subject groups” as used herein refers to any identifiable grouping within a genus or species, particularly one having a genetic component, e.g. strains, breeds, and ethnic groups. In an aspect, the genes are identified in a suitable taxon, including but not limited to murines, canines, felines, or hominids.

The particular pattern of change in gene expression for a panel of genes can serve as a profile of the effects of CR in an individual or group of subjects. This CR expression profile can in turn be used to identify substances and other treatments that mimic the effects of CR on gene expression. Such substances can therefore be expected to mimic other effects of CR. Therefore, CR-related gene panels and expression profiles can be used to screen substances for administration to subjects for the purpose of imparting the effects of CR to those subjects.

In one aspect of the present technology, the gene panels can represent broad genetic diversity, such that interpretation of results from an individual can be extrapolated to a large subject group. For example, an expression profile obtained from screening an ingredient in a particular strain of mouse can be used to predict similar effects of the ingredient in multiple strains of mice. In another example, a gene panel comprising genes identified in an individual belonging to a particular ethnic group can be used to screen for effective CR mimics across ethnic groups.

In an embodiment, more particular gene panels can comprise subsets of genes in the full panel described above. For example, one such panel can comprise CR-responsive genes that are more specific to tissues or tissue types. In another example, a subset of genes can be used that show more abundant expression under test conditions, or that are more or less sensitive to substance dosage. These criteria are not exhaustive of the factors by which genes for a specific panel may be selected so as to serve a particular purpose. In an aspect, the more specific panels can provide quicker and/or more readily interpretable results that can be utilized in an initial screening step to identify substances as candidates for testing against the full panel.

The gene panels and methods according to the present technology can be used in selecting components for formulations. In an embodiment, a method for determining if a candidate compound is likely to be useful in mimicking the effects of CR when administered to an animal can comprise treating an animal with a candidate compound; measuring expression of a plurality of genes from a CR gene panel; and determining whether the candidate compound mimics a CR expression profile of those genes. In a particular example, the CR expression profile can be obtained by analyzing tissues of an animal subjected to CR to measure the expression products of genes in the panel. The expression of those genes in the substance-treated animal can be compared to the CR expression profile to determine whether and to what degree the substance mimics CR. In one embodiment, the genes analyzed can be selected from a full panel of CR-responsive genes. In another embodiment, the genes analyzed are selected from a more specific panel. In a specific example, samples produced from a number of substances are initially screened by measuring expression of genes in a specific test panel. The substances are then ranked based on measurements or indices indicating the degree to which each substance mimics CR. In one aspect, the indices and ranking can be generated using conventional statistical tools. In another aspect, ranking can be done at least partly using a coding system that includes the treatment-induced fold change relative to the CR profile, the number of genes in the test panel, the number of genes significantly affected, or any combination thereof. A formulation can then be made by selecting one or more of the ranked substances. As a further validation step, the formulation or one or more of the substances can be tested against the full gene panel.

Compositions

Compositions for use in the diagnostic methods of the present invention include, but are not limited to, probes, amplification oligonucleotides, and antibodies. Particularly preferred compositions are useful for, necessary for, or sufficient for detecting the level of expression of one or more genes listed in Tables 1-6, from a biological sample (e.g. a sample of tissue) obtained from a subject of interest.

Any of these compositions, alone or in combination with other compositions of the present invention, may be provided in the form of a kit. For example, the single labeled probe and pair of amplification oligonucleotides may be provided in a kit for the amplification and detection and/or quantification of a panel of genes selected from a group consisting of the genes listed in Tables 1-6. The kit may include any and all components necessary or sufficient for assays including, but not limited to, the reagents themselves, buffers, control reagents (e.g., tissue samples, positive and negative control sample, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered.

### EXAMPLES

### Example 1

### Identification of Genes Differentially Expressed in CR and Control Subjects

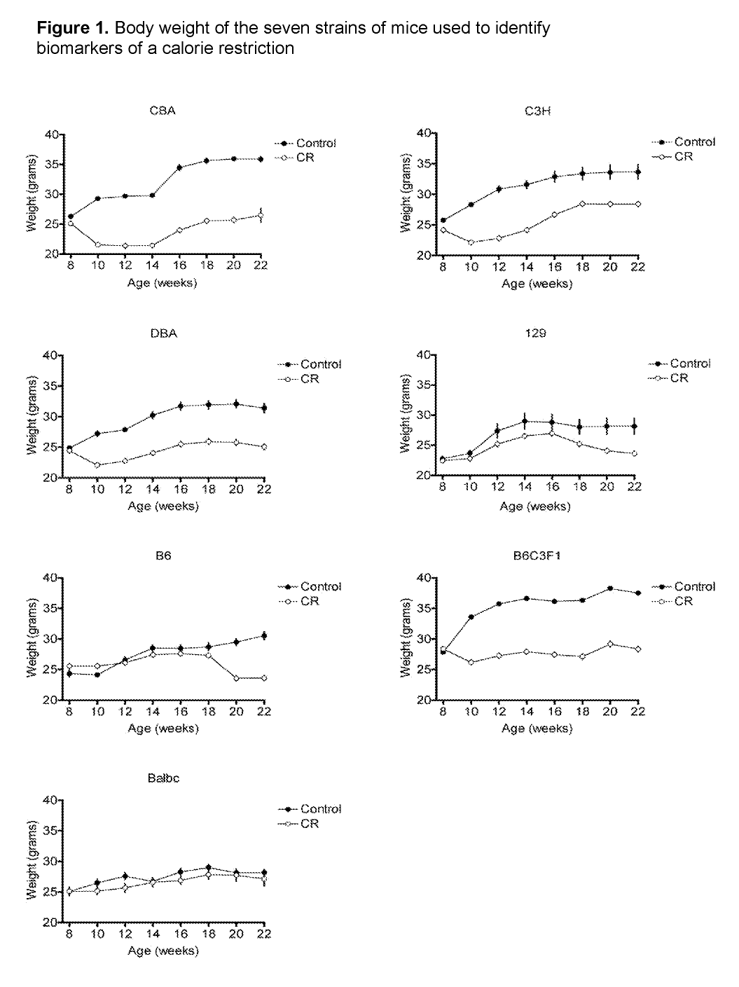

Experiments were conducted during the development of embodiments of the invention to identify genes which are differentially expressed in CR mice when compared to mice fed a control diet. Seven different strains of mice (129S1/SvImJ, C57BL/6J, BALB/cJ, C3H/HeJ, CBA/J, DBA/2J, and B6C3F1/J) were subjected to calorie restricted (CR) diets from two months until five months of age. Mice were fed a control diet based on the AIN93M formula or a diet with similar nutrient composition but representing 30-50% calorie restriction. Food allotments were tailored to the metabolism of each strain. The effects of the CR diets on body weights of each strain over the trial period are shown in FIG. 1. Tissues were collected from mice on the control and CR diets at 5 months of age. Gene expression levels of 20,789 genes were compared between CR and control mice. In heart tissue, 70 genes showed highly significant changes in gene expression in response to CR (see Table 1; p-value cutoff of <0.01 in four out of 7 strains and a >=1.2-fold or <=−1.2 fold change (FC) in expression in the C57BL/6J strain); in muscle tissue, 94 genes showed highly significant changes in gene expression in response to CR (see Table 2; p-value cutoff of <0.01 in five out of 7 strains and a >=1.3-fold or <=−1.3 FC in expression in the C57BL/6J strain); in white adipose tissue, 165 genes showed highly significant changes in gene expression in response to CR (see Table 3; p-value cutoff of <0.01 in six out of 7 strains and a >=1.5-fold or <=−1.5 FC in expression in the C57BL/6J strain).

TABLE 1
Heart Tissue

| | | FC in | |
|---|---|---|---|
| | | expression | # strains |
| | | (C57BL/ | with |
| | | 6J | significant |
| Gene symbol | Gene product | strain) | change |
| 1810013D10Rik | RIKEN cDNA 1810013D10 | 1.5 | 4 |
| | gene | | |
| 2700029M09Rik | RIKEN cDNA 2700029M09 | 1.3 | 5 |
| | gene | | |
| 5730494M16Rik | RIKEN cDNA 5730494M16 | 1.4 | 4 |
| | gene | | |
| Abhd10 | abhydrolase domain | 1.3 | 5 |
| | containing 10 | | |
| Alas1 | aminolevulinic acid | 2.2 | 5 |
| | synthase 1 | | |
| Aox1 | aldehyde oxidase 1 | −1.4 | 6 |
| Atp6v1d | ATPase, H+ transporting, | 1.4 | 4 |
| | lysosomal V1 subunit D | | |
| Calr3 | calreticulin 3 | −1.5 | 5 |
| Cat | catalase | −1.5 | 5 |
| Chrna2 | cholinergic receptor, | −1.2 | 4 |
| | nicotinic, alpha polypeptide | | |
| | 2 (neuronal) | | |
| Clock | circadian locomotor output | −1.5 | 4 |
| | cycles kaput | | |
| Cp | ceruloplasmin | −1.3 | 4 |
| Cry1 | cryptochrome 1 | 1.4 | 4 |
| | (photolyase-like) | | |
| Dhrs7c | dehydrogenase/reductase | 1.9 | 6 |
| | (SDR family) member 7C | | |
| Dnm1l | dynamin 1-like | 1.3 | 5 |
| Dtna | dystrobrevin alpha | 1.4 | 6 |
| Erbb2ip | Erbb2 interacting protein | −1.2 | 4 |
| Fbxl22 | F-box and leucine-rich | 1.7 | 4 |
| | repeat protein 22 | | |
| Gatc | glutamyl-tRNA(Gln) | 1.3 | 4 |
| | amidotransferase, subunit | | |
| | C homolog (bacterial) | | |
| Gbe1 | glucan (1,4-alpha-), | 1.2 | 4 |
| | branching enzyme 1 | | |
| Gclm | glutamate-cysteine ligase, | −1.4 | 5 |
| | modifier subunit | | |
| Grk5 | G protein-coupled receptor | −1.5 | 4 |
| | kinase 5 | | |
| Gsta3 | glutathione S-transferase, | 1.6 | 4 |
| | alpha 3 | | |
| Hccs | holocytochrome c | 1.3 | 4 |
| | synthetase | | |
| Hist1h1c | histone cluster 1, H1c | −1.4 | 4 |
| Hmgcs2 | 3-hydroxy-3-methylglutaryl- | 1.4 | 4 |
| | Coenzyme A synthase 2 | | |
| Hopx | HOP homeobox | 1.5 | 4 |
| Hus1 | Hus1 homolog (S. pombe) | 1.3 | 4 |
| ligp1 | interferon inducible GTPase 1 | −2.0 | 4 |
| Khdrbs3 | KH domain containing, | 1.2 | 4 |
| | RNA binding, signal | | |
| | transduction associated 3 | | |
| Krt222 | keratin 222 | 1.4 | 4 |
| Lyve1 | lymphatic vessel | 1.3 | 4 |
| | endothelial hyaluronan | | |
| | receptor 1 | | |
| Mdga1 | MAM domain containing | 1.3 | 4 |
| | glycosylphosphatidylinositol | | |
| | anchor 1 | | |
| Mpv17 | MpV17 mitochondrial inner | 1.3 | 5 |
| | membrane protein | | |
| Mrpl49 | mitochondrial ribosomal | 1.4 | 4 |
| | protein L49 | | |
| Parp14 | poly (ADP-ribose) | −1.3 | 4 |
| | polymerase family, member | | |
| | 14 | | |
| Pde1c | phosphodiesterase 1C | 1.3 | 4 |
| Pdhb | pyruvate dehydrogenase | 1.3 | 4 |
| | (lipoamide) beta | | |
| Pdss1 | prenyl (solanesyl) | 1.5 | 5 |
| | diphosphate synthase, | | |
| | subunit 1 | | |
| Perp | PERP, TP53 apoptosis | 1.6 | 5 |
| | effector | | |
| Pir | pirin | −1.4 | 4 |
| Plag1 | pleiomorphic adenoma | 1.4 | 4 |
| | gene 1 | | |
| Plbd1 | phospholipase B domain | −1.2 | 4 |
| | containing 1 | | |
| Polr3k | polymerase (RNA) III (DNA | 1.2 | 4 |
| | directed) polypeptide K | | |
| Prkag1 | protein kinase, AMP- | 1.2 | 5 |
| | activated, gamma 1 non- | | |
| | catalytic subunit | | |
| Prodh | proline dehydrogenase | 1.6 | 4 |
| Ptp4a1 | protein tyrosine | −1.3 | 4 |
| | phosphatase 4a1 | | |
| Rbx1 | ring-box 1 | 1.3 | 4 |
| Retsat | retinol saturase (all trans | −1.6 | 4 |
| | retinol 13,14 reductase) | | |
| Rnase1 | ribonuclease, RNase A | −1.2 | 5 |
| | family, 1 (pancreatic) | | |
| Rras2 | related RAS viral (r-ras) | −1.2 | 4 |
| | oncogene homolog 2 | | |
| Scd4 | stearoyl-coenzyme A | −3.5 | 6 |
| | desaturase 4 | | |
| Senp6 | SUMO/sentrin specific | −1.2 | 4 |
| | peptidase 6 | | |
| Sepx1 | selenoprotein X 1 | 1.3 | 4 |
| Slc31a1 | solute carrier family 31, | 1.2 | 5 |
| | member 1 | | |
| Slc39a10 | solute carrier family 39 | −1.4 | 4 |
| | (zinc transporter), member | | |
| | 10 | | |
| Slc40a1 | solute carrier family 40 | −1.5 | 5 |
| | (iron-regulated transporter), | | |
| | member 1 | | |
| Slc46a3 | solute carrier family 46, | 1.7 | 4 |
| | member 3 | | |
| Tfrc | transferrin receptor | 2.2 | 6 |
| Timm22 | translocase of inner | 1.3 | 4 |
| | mitochondrial membrane 22 | | |
| | homolog (yeast) | | |
| Tmx2 | thioredoxin-related | 1.5 | 4 |
| | transmembrane protein 2 | | |
| Ttll1 | tubulin tyrosine ligase-like 1 | 1.4 | 5 |
| Tuba4a | tubulin, alpha 4A | 4.3 | 5 |
| Tuba8 | tubulin, alpha 8 | 3.1 | 6 |
| Tubb2c | tubulin, beta 2C | 1.6 | 5 |
| Tubb6 | tubulin, beta 6 | 1.3 | 5 |
| Wfdc1 | WAP four-disulfide core | 1.5 | 4 |
| | domain 1 | | |
| Zdhhc4 | zinc finger, DHHC domain | 1.2 | 4 |
| | containing 4 | | |
| Zfyve21 | zinc finger, FYVE domain | 1.4 | 4 |
| | containing 21 | | |
| Zrsr1 | zinc finger (CCCH type), | −1.4 | 4 |
| | RNA binding motif and | | |
| | serine/arginine rich 1 | | |

TABLE 2
Skeletal Muscle Tissue

| | | FC in | # strains |
|---|---|---|---|
| | | expression | with |
| | | (C57BL/ | significant |
| Gene symbol | Gene product | 6J strain) | change |
| 4930451C15Rik | RIKEN cDNA 4930451C15 | −1.3 | 7 |
| | gene | | |
| Abcd2 | ATP-binding cassette, sub- | 1.4 | 6 |
| | family D (ALD), member 2 | | |
| Acot1 | acyl-CoA thioesterase 1 | −1.7 | 6 |
| Acot2 | acyl-CoA thioesterase 2 | −2.8 | 5 |
| Acsl1 | acyl-CoA synthetase long- | −1.5 | 5 |
| | chain family member 1 | | |
| Actc1 | actin, alpha, cardiac | 3.1 | 5 |
| | muscle 1 | | |
| AI317395 | expressed sequence | −1.5 | 5 |
| | AI317395 | | |
| Aimp2 | aminoacyl tRNA | 1.3 | 6 |
| | synthetase complex- | | |
| | interacting multifunctional | | |
| | protein 2 | | |
| Alg3 | asparagine-linked | 1.3 | 5 |
| | glycosylation 3 homolog | | |
| | (yeast, alpha-1,3- | | |
| | mannosyltransferase) | | |
| Angptl2 | angiopoietin-like 2 | 2.1 | 6 |
| Anxa7 | annexin A7 | −1.5 | 5 |
| Aqp4 | aquaporin 4 | 1.8 | 6 |
| Asb5 | ankyrin repeat and SOCs | −1.5 | 6 |
| | box-containing 5 | | |
| Cacnb1 | calcium channel, voltage- | 1.4 | 5 |
| | dependent, beta 1 subunit | | |
| Casp12 | caspase 12 | −2.0 | 5 |
| Cat | catalase | −1.4 | 6 |
| Cbr2 | carbonyl reductase 2 | −1.6 | 5 |
| Ccdc50 | coiled-coil domain | 1.3 | 5 |
| | containing 50 | | |
| Chrna2 | cholinergic receptor, | −1.4 | 6 |
| | nicotinic, alpha polypeptide | | |
| | 2 (neuronal) | | |
| Cnksr1 | connector enhancer of | −1.5 | 6 |
| | kinase suppressor of Ras 1 | | |
| Cntfr | ciliary neurotrophic factor | 2.0 | 6 |
| | receptor | | |
| Cntnap2 | contactin associated | 1.3 | 7 |
| | protein-like 2 | | |
| Crbn | cereblon | −1.5 | 5 |
| Dock4 | dedicator of cytokinesis 4 | 1.4 | 5 |
| Dynll1 | dynein light chain LC8-type 1 | 1.3 | 6 |
| Eda2r | ectodysplasin A2 receptor | −3.2 | 5 |
| Eif4ebp1 | eukaryotic translation | −1.3 | 5 |
| | initiation factor 4E binding | | |
| | protein 1 | | |
| Esr1 | estrogen receptor 1 (alpha) | 1.6 | 6 |
| Fam134b | family with sequence | −1.5 | 6 |
| | similarity 134, member B | | |
| Fam63a | family with sequence | −1.5 | 5 |
| | similarity 63, member A | | |
| Fam78a | family with sequence | 1.8 | 6 |
| | similarity 78, member A | | |
| Fhl3 | four and a half LIM | −1.4 | 6 |
| | domains 3 | | |
| Fzd7 | frizzled homolog 7 | −1.3 | 6 |
| | (Drosophila) | | |
| Gm15470 | predicted gene 15470 | −2.1 | 5 |
| Gm4980 | predicted gene 4980 | 1.3 | 5 |
| Gpd1 | glycerol-3-phosphate | 1.3 | 5 |
| | dehydrogenase 1 (soluble) | | |
| Grb10 | growth factor receptor | 1.4 | 6 |
| | bound protein 10 | | |
| Grem2 | gremlin 2 homolog, | 1.5 | 5 |
| | cysteine knot superfamily | | |
| | (Xenopus laevis) | | |
| Gstm2 | glutathione S-transferase, | 1.4 | 5 |
| | mu 2 | | |
| Hn1l | hematological and | 1.3 | 5 |
| | neurological expressed 1- | | |
| | like | | |
| Hr | hairless | 1.3 | 5 |
| Igf2 | insulin-like growth factor 2 | 1.4 | 5 |
| Ip6k3 | inositol hexaphosphate | 1.4 | 5 |
| | kinase 3 | | |
| Itgb5 | integrin beta 5 | −1.3 | 5 |
| Jph2 | junctophilin 2 | 1.4 | 5 |
| Kcnc4 | potassium voltage gated | 1.6 | 6 |
| | channel, Shaw-related | | |
| | subfamily, member 4 | | |
| Klf13 | Kruppel-like factor 13 | 1.5 | 6 |
| Lrrc2 | leucine rich repeat | −1.5 | 6 |
| | containing 2 | | |
| Lrrfip1 | leucine rich repeat (in FLII) | 1.6 | 6 |
| | interacting protein 1 | | |
| Mafa | v-maf musculoaponeurotic | 1.5 | 7 |
| | fibrosarcoma oncogene | | |
| | family, protein A (avian) | | |
| Map2k6 | mitogen-activated protein | 1.5 | 5 |
| | kinase kinase 6 | | |
| Mical2 | microtubule associated | 1.5 | 6 |
| | monoxygenase, calponin | | |
| | and LIM domain containing 2 | | |
| Mlec | malectin | 1.4 | 6 |
| Mlycd | malonyl-CoA | −1.9 | 5 |
| | decarboxylase | | |
| Myo5c | myosin VC | 1.3 | 5 |
| Npm1 | nucleophosmin 1 | −1.3 | 6 |
| Odc1 | ornithine decarboxylase, | −2.1 | 5 |
| | structural 1 | | |
| Palld | palladin, cytoskeletal | 1.9 | 6 |
| | associated protein | | |
| Parm1 | prostate androgen- | 1.4 | 5 |
| | regulated mucin-like | | |
| | protein 1 | | |
| Phlda3 | pleckstrin homology-like | −1.4 | 5 |
| | domain, family A, member 3 | | |
| Pip5k1a | phosphatidylinositol-4- | −1.6 | 5 |
| | phosphate 5-kinase, type 1 | | |
| | alpha | | |
| Pknox2 | Pbx/knotted 1 homeobox 2 | 1.5 | 6 |
| Plin5 | perilipin 5 | −1.8 | 6 |
| Plxna4 | plexin A4 | 1.3 | 5 |
| Ppard | peroxisome proliferator | 1.4 | 5 |
| | activator receptor delta | | |
| Ppp1r1a | protein phosphatase 1, | 1.3 | 6 |
| | regulatory (inhibitor) | | |
| | subunit 1A | | |
| Prune | prune homolog | −1.5 | 5 |
| | (Drosophila) | | |
| Psma1 | proteasome (prosome, | −1.3 | 6 |
| | macropain) subunit, alpha | | |
| | type 1 | | |
| Rabgef1 | RAB guanine nucleotide | −1.4 | 5 |
| | exchange factor (GEF) 1 | | |
| Retsat | retinol saturase (all trans | −1.7 | 5 |
| | retinol 13, 14 reductase) | | |
| Rnf114 | ring finger protein 114 | −1.3 | 5 |
| Rps7-ps2 | ribosomal protein S7, | −1.5 | 5 |
| | pseudogene 2 | | |
| Sel1l3 | sel-1 suppressor of lin-12- | 1.4 | 6 |
| | like 3 (C. elegans) | | |
| Serpine1 | serine (or cysteine) | −1.4 | 6 |
| | peptidase inhibitor, clade | | |
| | E, member 1 | | |
| Sgk1 | serum/glucocorticoid | 1.5 | 5 |
| | regulated kinase 1 | | |
| Sh3rf2 | SH3 domain containing | 2.2 | 5 |
| | ring finger 2 | | |
| Slc25a34 | solute carrier family 25, | −2.0 | 5 |
| | member 34 | | |
| Slc35f5 | solute carrier family 35, | −1.6 | 6 |
| | member F5 | | |
| Slc46a3 | solute carrier family 46, | 1.4 | 6 |
| | member 3 | | |
| Smtnl2 | smoothelin-like 2 | −1.3 | 5 |
| Sqstm1 | sequestosome 1 | −1.3 | 5 |
| Strbp | spermatid perinuclear RNA | 1.3 | 5 |
| | binding protein | | |
| Stxbp4 | syntaxin binding protein 4 | 1.7 | 6 |
| Tfrc | transferrin receptor | 1.3 | 6 |
| Thrsp | thyroid hormone | 1.5 | 5 |
| | responsive SPOT14 | | |
| | homolog (Rattus) | | |
| Tmem37 | transmembrane protein 37 | −1.7 | 5 |
| Tmem52 | transmembrane protein 52 | 1.3 | 6 |
| Tmx2 | thioredoxin-related | 1.3 | 6 |
| | transmembrane protein 2 | | |
| Trim35 | tripartite motif-containing | 1.5 | 6 |
| | 35 | | |
| Trim7 | tripartite motif-containing 7 | 1.3 | 5 |
| Ube2g1 | ubiquitin-conjugating | −1.3 | 6 |
| | enzyme E2G 1 (UBC7 | | |
| | homolog, C. elegans) | | |
| Ufsp1 | UFM1-specific peptidase 1 | 1.4 | 5 |
| Vldlr | very low density lipoprotein | −1.4 | 5 |
| | receptor | | |
| Ypel3 | yippee-like 3 (Drosophila) | −1.4 | 5 |

TABLE 3
White Adipose Tissue (WAT)

| | | FC in | # strains |
|---|---|---|---|
| | | expression | with |
| | | (C57BL/ | significant |
| Gene Symbol | Gene Title | 6J strain) | change |
| 2010003K11Rik | RIKEN cDNA 2010003K11 | 1.9 | 6 |
| | gene | | |
| 2310016C08Rik | RIKEN cDNA 2310016C08 | −2.0 | 6 |
| | gene | | |
| 4632428N05Rik | RIKEN cDNA 4632428N05 | −1.9 | 6 |
| | gene | | |
| 9630013D21Rik | RIKEN cDNA 9630013D21 | 2.7 | 6 |
| | gene | | |
| Aacs | acetoacetyl-CoA synthetase | 2.0 | 6 |
| Acaca | acetyl-Coenzyme A | 2.9 | 6 |
| | carboxylase alpha | | |
| Acly | ATP citrate lyase | 6.0 | 6 |
| Acss2 | acyl-CoA synthetase short- | 4.0 | 6 |
| | chain family member 2 | | |
| Acvr1b | activin A receptor, type 1B | −1.5 | 6 |
| Adap2 | ArfGAP with dual PH | −2.3 | 6 |
| | domains 2 | | |
| Adcy10 | adenylate cyclase 10 | 2.0 | 6 |
| Adcy5 | adenylate cyclase 5 | −2.9 | 6 |
| Adi1 | acireductone dioxygenase 1 | 1.6 | 6 |
| Agpat2 | 1-acylglycerol-3-phosphate | 1.7 | 6 |
| | O-acyltransferase 2 | | |
| | (lysophosphatidic acid | | |
| | acyltransferase, beta) | | |
| Aifm1 | apoptosis-inducing factor, | 1.7 | 6 |
| | mitochondrion-associated 1 | | |
| Ak2 | adenylate kinase 2 | 1.6 | 6 |
| Aldoa | aldolase A, fructose- | 1.5 | 6 |
| | bisphosphate | | |
| Ank | progressive ankylosis | 1.5 | 6 |
| Anpep | alanyl (membrane) | −1.5 | 6 |
| | aminopeptidase | | |
| Apobec1 | apolipoprotein B mRNA | −1.7 | 6 |
| | editing enzyme, catalytic | | |
| | polypeptide 1 | | |
| Atf3 | activating transcription | −1.7 | 6 |
| | factor 3 | | |
| Atp5f1 | ATP synthase, H+ | 1.5 | 6 |
| | transporting, mitochondrial | | |
| | F0 complex, subunit B1 | | |
| Aven | apoptosis, caspase | 1.5 | 6 |
| | activation inhibitor | | |
| Brp44 | brain protein 44 | 1.8 | 6 |
| Brp44l | brain protein 44-like | 1.6 | 6 |
| C1qtnf1 | C1q and tumor necrosis | −1.5 | 6 |
| | factor related protein 1 | | |
| C3ar1 | complement component 3a | −1.7 | 6 |
| | receptor 1 | | |
| Ccl9 | chemokine (C-C motif) | −1.6 | 6 |
| | ligand 9 | | |
| Cd300a | CD300A antigen | −1.5 | 6 |
| Cd68 | CD68 antigen | −1.9 | 6 |
| Cdk5 | cyclin-dependent kinase 5 | 1.6 | 6 |
| Cdkn2c | cyclin-dependent kinase | 2.4 | 6 |
| | inhibitor 2C (p18, inhibits | | |
| | CDK4) | | |
| Cfb | complement factor B | −1.9 | 6 |
| Chchd10 | coiled-coil-helix-coiled-coil- | 1.6 | 6 |
| | helix domain containing 10 | | |
| Clec12a | C-type lectin domain family | −1.5 | 6 |
| | 12, member a | | |
| Clec2d | C-type lectin domain family | −1.5 | 6 |
| | 2, member d | | |
| Coasy | Coenzyme A synthase | 1.9 | 6 |
| Coq7 | demethyl-Q 7 | 1.5 | 6 |
| Creb3l1 | cAMP responsive element | −1.6 | 6 |
| | binding protein 3-like 1 | | |
| Cs | citrate synthase | 1.5 | 6 |
| Cxcl12 | chemokine (C—X—C motif) | −1.7 | 6 |
| | ligand 12 | | |
| Cxx1c | CAAX box 1 homolog C | −1.8 | 6 |
| | (human) | | |
| Cyb5b | cytochrome b5 type B | 2.1 | 6 |
| Cyc1 | cytochrome c-1 | 1.5 | 6 |
| Dhrs7 | dehydrogenase/reductase | 2.1 | 6 |
| | (SDR family) member 7 | | |
| Dlat | dihydrolipoamide S- | 1.8 | 6 |
| | acetyltransferase (E2 | | |
| | component of pyruvate | | |
| | dehydrogenase complex) | | |
| Dram1 | DNA-damage regulated | −1.6 | 6 |
| | autophagy modulator 1 | | |
| Dusp9 | dual specificity phosphatase 9 | −2.1 | 6 |
| Ear2 | eosinophil-associated, | −2.1 | 6 |
| | ribonuclease A family, | | |
| | member 2 | | |
| Elovl6 | ELOVL family member 6, | 10.7 | 6 |
| | elongation of long chain fatty | | |
| | acids (yeast) | | |
| Emp1 | epithelial membrane protein 1 | −2.3 | 6 |
| Enc1 | ectodermal-neural cortex 1 | −2.8 | 6 |
| Enpep | glutamyl aminopeptidase | 2.0 | 6 |
| Ergic1 | endoplasmic reticulum-golgi | 1.5 | 6 |
| | intermediate compartment | | |
| | (ERGIC) 1 | | |
| Fam198b | family with sequence | −1.6 | 6 |
| | similarity 198, member B | | |
| Fam57b | family with sequence | 1.9 | 6 |
| | similarity 57, member B | | |
| Fasn | fatty acid synthase | 2.4 | 6 |
| Fbxo21 | F-box protein 21 | −1.5 | 6 |
| Fdft1 | farnesyl diphosphate | 2.0 | 6 |
| | farnesyl transferase 1 | | |
| Fdps | farnesyl diphosphate | 3.0 | 6 |
| | synthetase | | |
| Fn3k | fructosamine 3 kinase | 1.5 | 6 |
| Gbe1 | glucan (1,4-alpha-), | 2.1 | 6 |
| | branching enzyme 1 | | |
| Gcnt2 | glucosaminyl (N-acetyl) | −2.0 | 6 |
| | transferase 2, I-branching | | |
| | enzyme | | |
| Gm6560 | predicted gene 6560 | 1.5 | 6 |
| Gpd1 | glycerol-3-phosphate | 1.8 | 6 |
| | dehydrogenase 1 (soluble) | | |
| Gpd2 | glycerol phosphate | 2.5 | 6 |
| | dehydrogenase 2, | | |
| | mitochondrial | | |
| Gpr156 | G protein-coupled receptor | −1.6 | 6 |
| | 156 | | |
| Gpx1 | glutathione peroxidase 1 | −1.7 | 6 |
| Gys2 | glycogen synthase 2 | 3.2 | 6 |
| Hmgcs1 | 3-hydroxy-3-methylglutaryl- | −1.7 | 6 |
| | Coenzyme A synthase 1 | | |
| Hmgcs2 | 3-hydroxy-3-methylglutaryl- | −2.7 | 6 |
| | Coenzyme A synthase 2 | | |
| Hpgds | hematopoietic prostaglandin | −1.6 | 6 |
| | D synthase | | |
| Hr | hairless | −1.8 | 6 |
| Hspd1 | heat shock protein 1 | 1.5 | 6 |
| | (chaperonin) | | |
| Htra3 | HtrA serine peptidase 3 | −2.0 | 6 |
| Idh3a | isocitrate dehydrogenase 3 | 1.5 | 6 |
| | (NAD+) alpha | | |
| Ifi27l2a | interferon, alpha-inducible | −3.4 | 6 |
| | protein 27 like 2A | | |
| Kcmf1 | potassium channel | 1.5 | 6 |
| | modulatory factor 1 | | |
| Lhfpl2 | lipoma HMGIC fusion | −1.9 | 6 |
| | partner-like 2 | | |
| Lilrb4 | leukocyte immunoglobulin- | −1.9 | 6 |
| | like receptor, subfamily B, | | |
| | member 4 | | |
| Lrg1 | leucine-rich alpha-2- | −1.6 | 6 |
| | glycoprotein 1 | | |
| Lss | lanosterol synthase | 2.7 | 6 |
| Ltbp3 | latent transforming growth | −1.5 | 6 |
| | factor beta binding protein 3 | | |
| Me1 | malic enzyme 1, NADP(+)- | 2.9 | 6 |
| | dependent, cytosolic | | |
| Mlxipl | MLX interacting protein-like | 1.9 | 6 |
| Mmp12 | matrix metallopeptidase 12 | −3.6 | 6 |
| Mmp14 | matrix metallopeptidase 14 | −1.5 | 6 |
| | (membrane-inserted) | | |
| Mogat2 | monoacylglycerol O- | 4.5 | 6 |
| | acyltransferase 2 | | |
| Mpeg1 | macrophage expressed | −2.4 | 6 |
| | gene 1 | | |
| Mpi | mannose phosphate | 1.5 | 6 |
| | isomerase | | |
| Mrpl48 | mitochondrial ribosomal | 1.5 | 6 |
| | protein L48 | | |
| Mrps22 | mitochondrial ribosomal | 1.5 | 6 |
| | protein S22 | | |
| Ms4a7 | membrane-spanning 4- | −1.6 | 6 |
| | domains, subfamily A, | | |
| | member 7 | | |
| Mtch2 | mitochondrial carrier | 1.6 | 6 |
| | homolog 2 (C. elegans) | | |
| Nampt | nicotinamide | 2.0 | 6 |
| | phosphoribosyltransferase | | |
| Nap1l1 | nucleosome assembly | −1.5 | 6 |
| | protein 1-like 1 | | |
| Nav1 | neuron navigator 1 | −1.7 | 6 |
| Ndufs4 | NADH dehydrogenase | 1.5 | 6 |
| | (ubiquinone) Fe—S protein 4 | | |
| Ntrk2 | neurotrophic tyrosine kinase, | −1.6 | 6 |
| | receptor, type 2 | | |
| Oscp1 | organic solute carrier | 2.0 | 6 |
| | partner 1 | | |
| Parm1 | prostate androgen-regulated | 2.9 | 6 |
| | mucin-like protein 1 | | |
| Pde1b | phosphodiesterase 1B, | −1.6 | 6 |
| | Ca2+-calmodulin dependent | | |
| Pdhb | pyruvate dehydrogenase | 2.0 | 6 |
| | (lipoamide) beta | | |
| Pdhx | pyruvate dehydrogenase | 1.5 | 6 |
| | complex, component X | | |
| Peg3 | paternally expressed 3 | −3.6 | 6 |
| Pex16 | peroxisomal biogenesis | 1.5 | 6 |
| | factor 16 | | |
| Pfkfb3 | 6-phosphofructo-2- | −2.1 | 6 |
| | kinase/fructose-2,6- | | |
| | biphosphatase 3 | | |
| Pgd | phosphogluconate | 1.6 | 6 |
| | dehydrogenase | | |
| Pgk1 | phosphoglycerate kinase 1 | 1.5 | 6 |
| Plekho1 | pleckstrin homology domain | −1.5 | 6 |
| | containing, family O | | |
| | member 1 | | |
| Pmepa1 | prostate transmembrane | −1.6 | 6 |
| | protein, androgen induced 1 | | |
| Pmvk | phosphomevalonate kinase | 1.8 | 6 |
| Polr3g | polymerase (RNA) III (DNA | 1.6 | 6 |
| | directed) polypeptide G | | |
| Pomt1 | protein-O- | 1.6 | 6 |
| | mannosyltransferase 1 | | |
| Ppap2b | phosphatidic acid | −1.5 | 6 |
| | phosphatase type 2B | | |
| Prkd1 | protein kinase D1 | 1.6 | 6 |
| Prps1 | phosphoribosyl | −1.7 | 6 |
| | pyrophosphate synthetase 1 | | |
| Psmd12 | proteasome (prosome, | 1.5 | 6 |
| | macropain) 26S subunit, | | |
| | non-ATPase, 12 | | |
| Psmd14 | proteasome (prosome, | 1.5 | 6 |
| | macropain) 26S subunit, | | |
| | non-ATPase, 14 | | |
| Ptges | prostaglandin E synthase | 1.7 | 6 |
| Pth1r | parathyroid hormone 1 | 1.5 | 6 |
| | receptor | | |
| Ptrf | polymerase I and transcript | −1.6 | 6 |
| | release factor | | |
| Ptrh2 | peptidyl-tRNA hydrolase 2 | 1.6 | 6 |
| Pygl | liver glycogen phosphorylase | 1.9 | 6 |
| Ralgapa2 | Ral GTPase activating | 1.6 | 6 |
| | protein, alpha subunit 2 | | |
| | (catalytic) | | |
| Ramp2 | receptor (calcitonin) activity | −1.5 | 6 |
| | modifying protein 2 | | |
| Rbm38 | RNA binding motif protein 38 | 1.8 | 6 |
| Sbk1 | SH3-binding kinase 1 | 1.7 | 6 |
| Sctr | secretin receptor | −2.6 | 6 |
| Serpine1 | serine (or cysteine) | −3.7 | 6 |
| | peptidase inhibitor, clade E, | | |
| | member 1 | | |
| Sesn2 | sestrin 2 | −2.1 | 6 |
| Setd8 | SET domain containing | 1.5 | 6 |
| | (lysine methyltransferase) 8 | | |
| Sfrp5 | secreted frizzled-related | −3.8 | 6 |
| | sequence protein 5 | | |
| Sh3pxd2a | SH3 and PX domains 2A | −1.9 | 6 |
| Sh3tc1 | SH3 domain and | −1.8 | 6 |
| | tetratricopeptide repeats 1 | | |
| Shisa6 | shisa homolog 6 (Xenopus | 1.5 | 6 |
| | laevis) | | |
| Slc11a1 | solute carrier family 11 | −1.7 | 6 |
| | (proton-coupled divalent | | |
| | metal ion transporters), | | |
| | member 1 | | |
| Slc25a1 | solute carrier family 25 | 2.1 | 6 |
| | (mitochondrial carrier, citrate | | |
| | transporter), member 1 | | |
| Slc25a10 | solute carrier family 25 | 2.5 | 6 |
| | (mitochondrial carrier, | | |
| | dicarboxylate transporter), | | |
| | member 10 | | |
| Slc25a11 | solute carrier family 25 | 1.5 | 6 |
| | (mitochondrial carrier | | |
| | oxoglutarate carrier), | | |
| | member 11 | | |
| Slc25a35 | solute carrier family 25, | 2.3 | 6 |
| | member 35 | | |
| Slc2a4 | solute carrier family 2 | 1.8 | 6 |
| | (facilitated glucose | | |
| | transporter), member 4 | | |
| Slc2a5 | solute carrier family 2 | 3.7 | 6 |
| | (facilitated glucose | | |
| | transporter), member 5 | | |
| Slc9a3r2 | solute carrier family 9 | −1.6 | 6 |
| | (sodium/hydrogen | | |
| | exchanger), member 3 | | |
| | regulator 2 | | |
| Slco2b1 | solute carrier organic anion | −1.6 | 6 |
| | transporter family, member | | |
| | 2b1 | | |
| Sod2 | superoxide dismutase 2, | 1.5 | 6 |
| | mitochondrial | | |
| Sorl1 | sortilin-related receptor, | 3.1 | 6 |
| | LDLR class A repeats- | | |
| | containing | | |
| Srgap1 | SLIT-ROBO Rho GTPase | −1.5 | 6 |
| | activating protein 1 | | |
| Srpx2 | sushi-repeat-containing | −2.1 | 6 |
| | protein, X-linked 2 | | |
| Svep1 | sushi, von Willebrand factor | −1.5 | 6 |
| | type A, EGF and pentraxin | | |
| | domain containing 1 | | |
| Thbs1 | thrombospondin 1 | −7.5 | 6 |
| Thrsp | thyroid hormone responsive | 2.7 | 6 |
| | SPOT14 homolog (Rattus) | | |
| Tkt | transketolase | 2.6 | 6 |
| Tlcd1 | TLC domain containing 1 | 2.6 | 6 |
| Tmem53 | transmembrane protein 53 | 1.9 | 6 |
| Tnfrsf1b | tumor necrosis factor | −1.5 | 6 |
| | receptor superfamily, | | |
| | member 1b | | |
| Tpi1 | triosephosphate isomerase 1 | 1.5 | 6 |
| Trp53inp2 | transformation related | −1.6 | 6 |
| | protein 53 inducible nuclear | | |
| | protein 2 | | |
| Ttc25 | tetratricopeptide repeat | 1.5 | 6 |
| | domain 25 | | |
| Tubg1 | tubulin, gamma 1 | 1.6 | 6 |
| Tusc5 | tumor suppressor candidate 5 | −1.6 | 6 |
| Unc5a | unc-5 homolog A (C. elegans) | 2.9 | 6 |
| Uxt | ubiquitously expressed | 1.6 | 6 |
| | transcript | | |
| Vnn1 | vanin 1 | −3.0 | 6 |
| Wwtr1 | WW domain containing | −1.5 | 6 |
| | transcription regulator 1 | | |

In order to generate a panel of markers that can be used for RT-PCR analysis in heart tissue, eight potential markers of CR from Table 1 were selected for confirmation of array data by RT-PCR (Table 4). Genes were selected based on multiple factors including (but not limited to): abundant expression in the microarray experiment, robust change in gene expression in response to CR, and/or previous association with metabolic pathways affected by a CR diet. Using the RNA samples from a separate cohort of control and CR C57BL/6J mice than those used in the array study, quantitative RT-PCR analysis revealed that all genes were significantly changed by CR.

TABLE 4
Heart Tissue

| Gene | Gene Product | Fold change | p-value |
|---|---|---|---|
| Alas1 | aminolevulinic acid synthase 1 | 2.3 | <0.0001 |
| Aox1 | aldehyde oxidase 1 | −2.8 | 0.0001 |
| Cat | Catalase | −2.5 | 0.0003 |
| Chrna2 | cholinergic receptor, nicotinic, alpha | −8.3 | <0.0001 |
| | polypeptide 2 (neuronal) | | |
| Retsat | retinol saturase (all trans retinol 13, 14 | −2.5 | <0.0001 |
| | reductase) | | |
| Scd4 | stearoyl-coenzyme A desaturase 4 | −8.3 | <0.0001 |
| Tfrc | transferrin receptor | 2.1 | 0.0313 |
| Tuba8 | tubulin, alpha 8 | 2.0 | 0.0008 |

In order to generate a panel of markers that can be used for RT-PCR analysis in muscle tissue, ten potential markers of CR from Table 2 were selected for confirmation of array data by RT-PCR (Table 5). Genes were selected based on multiple factors including (but not limited to): abundant expression in the microarray experiment, robust change in gene expression in response to CR, and/or previous association with metabolic pathways affected by a CR diet. Using the RNA samples from a separate cohort of C57BL/6J mice than those used in the array study, quantitative RT-PCR analysis revealed that all genes were significantly changed by CR.

TABLE 5
Skeletal Muscle Tissue

| Gene | Gene Product | Fold change | p-value |
|---|---|---|---|
| Acot2 | acyl-CoA thioesterase 2 | −4.1 | <0.0001 |
| Actc1 | actin, alpha, cardiac muscle 1 | 7.8 | <0.0001 |
| Cat | Catalase | −1.8 | <0.0001 |
| Chrna2 | cholinergic receptor, nicotinic, alpha | −2.1 | 0.0012 |
| | polypeptide 2 (neuronal) | | |
| Cntfr | ciliary neurotrophic factor receptor | 2.7 | <0.0001 |
| Cntnap2 | contactin associated protein-like 2 | 4.2 | 0.0001 |
| Esr1 | estrogen receptor 1 (alpha) 6/7 | 2.7 | 0.0005 |
| Kcnc4 | potassium voltage gated channel, | 2.5 | <0.0001 |
| | Shaw-related subfamily, member 4 | | |
| Mlycd | malonyl-CoA decarboxylase | −2.1 | <0.0001 |
| Sgk1 | serum/glucocorticoid regulated kinase 1 | 2.2 | <0.0001 |

In order to generate a panel of markers that can be used for RT-PCR analysis in white adipose tissue, fifteen potential markers of CR from Table 3 were selected for confirmation of array data by RT-PCR (Table 6). Genes were selected based on multiple factors including (but not limited to): abundant expression in the microarray experiment, robust change in gene expression in response to CR, and/or previous association with metabolic pathways affected by a CR diet. Using the RNA samples from a separate cohort of C57BL/6J mice than those used in the array study, quantitative RT-PCR analysis revealed that all genes were significantly changed by CR.

TABLE 6
White Adipose Tissue

| | | Fold | |
|---|---|---|---|
| Gene | Gene Product | change | p-value |
| Acaca | acetyl-Coenzyme A carboxylase alpha | 9.5 | <0.0001 |
| Acss2 | acyl-CoA synthetase short-chain family | 14.9 | <0.0001 |
| | member 2 | | |
| Cd68 | CD68 antigen | −3.4 | <0.0001 |
| Cfb | complement factor B | −3.0 | <0.0001 |
| Gpd2 | glycerol phosphate dehydrogenase 2, | 5.3 | <0.0001 |
| | mitochondrial | | |
| Ifi27l2a | Ifi27l2a interferon, alpha-inducible protein | −3.4 | <0.0001 |
| | 27 like 2A | | |
| Me1 | malic enzyme 1, NADP(+)-dependent, | 10.8 | <0.0001 |
| | cytosolic | | |
| Nampt | nicotinamide phosphoribosyltransferase | 2.0 | 0.0066 |
| Ndufs4 | NADH dehydrogenase (ubiquinone) | 2.1 | <0.0001 |
| | Fe—S protein 4 | | |
| Parm1 | prostate androgen-regulated mucin-like | 13.1 | <0.0001 |
| | protein 1 | | |
| Serpine1 | serine (or cysteine) peptidase inhibitor, | −17.7 | <0.0001 |
| | clade E, member 1 | | |
| Slc2a4 | solute carrier family 2 (facilitated glucose | 2.8 | 0.0003 |
| | transporter), member 4 | | |
| Srpx2 | sushi-repeat-containing protein, X-linked 2 | −3.5 | <0.0001 |
| Thbs1 | thrombospondin 1 | −11.5 | <0.0001 |
| Tkt | Transketolase | 8.3 | <0.0001 |

### Example 2

### Testing Ingredients for Mimicry of Caloric Restriction

Feeding Study:

C57BL/6J mice were purchased from Jackson Laboratories at 6 weeks of age and maintained as described previously in Barger J L, et al. (2008) A Low Dose of Dietary Resveratrol Partially Mimics Caloric Restriction and Retards Aging Parameters in Mice. PLoS ONE 3(6): e2264 (http://dx.doi.org/10.1371/journal.pone.0002264). Briefly, mice were individually housed in shoebox cages and provided with 24 grams (˜84 kcal) of AIN-93M diet per week (7 grams on Monday and Wednesday and 10 grams on Friday). Starting at 8 weeks of age and continuing until 22 weeks of age, mice were either a) maintained on the AIN-93M diet (control group), b) fed a Calorie Restricted (CR) diet providing 63 kcal/week of a modified AIN93M from 8-16 weeks of age and then further reduced to a diet providing 49 kcal/week of a modified AIN93M from 16-22 weeks of age; or c) were assigned to an AIN93M diet supplemented with one of the following test ingredients: 1) bezafibrate at a dose of 5,000 mg/kg diet; 2) metformin at a dose of 1,909 mg/kg diet; 3) L-carnitine at a dose of 1,800 mg/kg diet; 4) blood orange extract at a dose of 18 mg/kg of body weight; 5) purple corn extract at a dose of 22 mg/kg of body weight; 6) resveratrol at a dose of 30 mg/kg of body weight; and 7) quercetin at a dose of 17.6 mg/kg of body weight. At 22 weeks of age, tissues were collected from the mice, flash-frozen in liquid nitrogen and stored at −80° C. for later analysis.

In order to screen the ingredients for their ability to mimic CR, quantitative real-time PCR (RT-qPCR) analysis was performed on RNA isolated from white adipose tissue from all groups of mice. Experimental methods and data analysis for RT-qPCR experiments have been published previously in Barger, J L et al., (2008) Short-term consumption of a resveratrol-containing nutraceutical mixture mimics gene expression of long-term caloric restriction in mouse heart, Exp. Gerontology 43(9):859 (http://dx.doi.org/10.1016/j.exger.2008.06.013). Briefly, the magnitude of change in gene expression was determined for each of the genes listed in Table 6 for the CR and treatment groups compared to the control animals. Two-tailed t-tests (assuming equal variance) were used to determine if the change in expression for individual genes was statistically significant. The magnitude of the change in expression (“fold change”) values were log₂-adjusted to fit normality assumptions for statistical analyses.

Results

CR mimicry was expressed as the fold change observed in each gene for the test group as a percentage of the fold change observed for that gene in the CR group. Table 7 shows the CR mimicry achieved by each ingredient for each gene that was significantly changed by that ingredient.

TABLE 7

| | Acaca | Acss2 | Cd68 | Gpd2 | Ifi27I2a | Me1 | Nampt | Ndufs4 | Parm1 | Serpine1 | Slc2a4 |
|---|---|---|---|---|---|---|---|---|---|---|---|
| Bezafibrate | 59% | 21% | 58% | 93% | 75% | 24% | 233% | 108% | 47% | — | 113% |
| Metformin | 17% | — | — | 27% | — | 11% | — | — | 10% | — | — |
| L- | 5% | — | — | 15% | — | — | 84% | 40% | 3% | 52% | 16% |
| carnitine | | | | | | | | | | | |
| Blood | 8% | — | — | 11% | 54% | — | 75% | 22% | 3% | — | 19% |
| Orange | | | | | | | | | | | |
| Extract | | | | | | | | | | | |
| Purple | 10% | — | — | — | — | 5% | 76% | — | — | 48% | — |
| Corn | | | | | | | | | | | |
| Extract | | | | | | | | | | | |
| Resveratrol | 8% | — | — | 10% | — | — | 65% | — | — | 55% | — |
| Quercetin | — | — | — | 5% | — | — | — | — | — | — | 35% |

Ranking CR Mimetic Ingredients

The ingredients tested were based on their mimetic effect across the gene panel. The mimicry values for all of the significantly changed genes were averaged for each ingredient.

In one ranking approach, the average mimicry (as a fraction of CR) was multiplied by the number of significantly changed genes to obtain a mimetic index (CMII). As shown in Table 8, bezafibrate was most effective in mimicking CR, while quercetin showed the lowest degree of CR mimicry.

Another ranking approach was used to reflect effects across all genes. A CR Mimetic Index (CRMI) was calculated per gene for each ingredient by assigning each a number of points based on its mimicry score for each gene. Positive points were given for positive mimicry values (11 to 20%=1 point, 21 to 30%=2 points, 31 to 40%=3 points, and so on). Negative mimicry values (i.e. where the gene expression effect was opposite that observed for CR) received corresponding negative scores (i.e. −11 to −20%=−1 point, −21 to −30%=−2 points, −31 to −40%=3 points, and so on). Five points were added for statistical significance of a mimicry value. The average CRMI for each ingredient is shown in Table 8.

TABLE 8

| | No. of | | | |
|---|---|---|---|---|
| | genes | | | |
| | significantly | Avg. | | Avg. |
| | changed | mimicry | CMII | CRMI |
| Bezafibrate | 10 | 83% | 8.3 | 128 |
| L-carnitine | 7 | 31% | 2.2 | 55 |
| Blood | 7 | 28% | 1.93 | 52 |
| Orange | | | | |
| Extract | | | | |
| Purple | 4 | 35% | 1.39 | 37 |
| Corn | | | | |
| Extract | | | | |
| Resveratrol | 4 | 34% | 1.38 | 35 |
| Metformin | 4 | 16% | 0.6 | 31 |
| Quercetin | 2 | 20% | 0.4 | 17 |

### Example 3

### Testing Effects of Dose on CR Mimicry by Bezafibrate

In the experimental protocol of Example 2, bezafibrate at 5,000 mg/kg diet was also compared to lower doses (100 and 500 mg/kg). Table 9 shows the degree of CR mimicry achieved by each dosage for each gene that was significantly changed by that dosage.

TABLE 9

| Beza | | | | | | | | | | | |
|---|---|---|---|---|---|---|---|---|---|---|---|
| dosage, | | | | | | | | | | | |
| mg/kg | | | | | | | | | | | |
| diet | Acaca | Acss2 | Cd68 | Gpd2 | Ifi27I2a | Me1 | Nampt | Ndufs4 | Parm1 | Serpine1 | Slc2a4 |
| 5000 | 59% | 21% | 58% | 93% | 75% | 24% | 233% | 108% | 47% | — | 113% |
| 500 | 23% | 17% | — | 31% | — | 18% | 87% | — | 19% | — | 69% |
| 100 | 20% | — | 58% | 23% | 39% | — | — | — | — | — | — |

Mimetic indices were calculated for the 500 mg/kg and 100 mg/kg doses as in Example 2. As shown in Table 10, the degree to which the bezafibrate mimicked CR was dose-dependent.

TABLE 10

| | No. of | | | |
|---|---|---|---|---|
| Bezafibrate | genes | | | |
| dosage, | significantly | Avg. | | Avg. |
| mg/kg diet | changed | Mimicry | CMII | CRMI |
| 5000 | 10 | 83% | 8.3 | 128 |
| 500 | 7 | 38% | 2.6 | 68 |
| 100 | 4 | 35% | 1.4 | 28 |

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

1-11. (canceled)

12. A probe for detecting differential expression of universal markers of CR in a tissue, comprising one of:
a) a polynucleotide that specifically hybridizes a gene listed in Tables 1 through 3 or a fragment thereof; or
b) a polypeptide binding agent that binds to a polypeptide encoded by a gene listed in Tables 1 through 6.

13. The probe of claim 12, wherein the tissue is heart tissue and the probes comprise polynucleotides selected from the genes listed in Table 1.

14. The probe of claim 13, wherein the polynucleotides are selected from genes listed in Table 4.

15. The probe of claim 12, wherein the tissue is skeletal muscle and the probes comprise polynucleotides selected from Table 2.

16. The probe of claim 15, wherein the polynucleotides are selected from genes listed in Table 5.

17. The probe of claim 12, wherein the tissue is white adipose and the probes comprise polynucleotides selected from Table 3.

18. The probe of claim 17, wherein the polynucleotides are selected from genes listed in Table 6.

19-22. (canceled)
